(19) United States
(12) Patent Application Publication
(10) Pub. No.: US 2007/0155661 A1
(43) Pub. Date: Jul. 5, 2007

(54) Methods and compositions for modulating the development of stem cells

(75) Inventor: Seung Kim, Stanford, CA (US)

Correspondence Address: FISH & NEAVE IP GROUP;ROPES & GRAY LLP, ONE INTERNATIONAL PLACE, BOSTON, MA, 02110-2624, US

(73) Assignee: THE BOARD of TRUSTEES of the LELAND STANDORD JUNIOR UNIVERSITY, PALO ALTO, CA (US)

(21) Appl. No.: 10/545,582

(22) PCT Filed: Feb. 17, 2004
(86) PCT No.: PCT/US04/04639

§ 371 Date: Nov. 28, 2006

Related U.S. Application Data
(60) Provisional application No. 60/447,673, filed on Feb. 14, 2003.

(51) Int. Cl. A61K 38/18; A61K 38/17; G01N 33/53; C12P 21/06; A61K 38/28

(52) U.S. Cl. 514/12; 435/7.1; 435/69.1; 530/303

(57) ABSTRACT

The application provides, among other things, methods for increasing production of insulin in cells by contacting the cells with a BMP family member and/or an activator of a BMP pathway. The application also provides methods for increasing the production of beta cell precursor cells by contacting suitable cells with an inhibitor of a BMP pathway.

### CROSS-REFERENCE TO RELATED APPLICATIONS

This application claims the benefit of the filing date of U.S. Provisional Patent Application Ser. No. 60/447,673 entitled “Methods and Compositions for Modulating the Development of Stem Cells” and listing Seung Kim as inventor. The aforementioned provisional application is incorporated herein in its entirety.

### BACKGROUND

Diabetes mellitus (DM) is a major cause of morbidity and mortality worldwide, and incidence rates of type I and type II DM are increasing. In type I DM, destruction of insulin-producing pancreatic islets leads to a prolonged illness often culminating in devastating multisystem organ failure and early mortality. Clinical trials demonstrate that tight glucose regulation can prevent the development of diabetic complications, but attempts to achieve this regulation by exogenous insulin administration are only partially successful.

Recent evidence suggests that islet cell transplantation with improved systemic immunosuppression may provide a short-term durable remission in insulin requirements in type I diabetics (Shapiro et al, 2000, N Engl J Med. 343: 230-238; Ryan et al, 2001, Diabetes 50: 710-719). However, in DM and the vast majority of other human diseases amenable to treatment by tissue replacement, there is an extreme shortage of engraftable donor tissues. An expandable source of tissues like human stem cells may provide the best promise for tissue replacement strategies for human diseases.

Stem cells, including embryonic stem (ES) cells and various adult stem cells provide a promising potential means for cell-replacement therapy in human diseases. Stem cells may provide serve as an inexhaustible source for the production of replacement islets for transplantation in diabetic humans. However, conditions to produce stably-differentiated functional insulin-producing cell compositions (ICCs) with stem cells generally, and particularly ES stem cells, have not been developed to a clinically satisfactory level.

Methods to provide a renewable source of replacement islets from stem cells could transform therapeutics in DM. Likewise, methods for stimulating the production of insulin producing cells in patients could also have significant therapeutic effects.

### SUMMARY

In certain aspects, the application provides methods for manipulating the development of insulin producing cells and beta cell precursor cells by activating or antagonizing a BMP signaling pathway. Such methods may be applied, for example, to cultured cells or to subjects in need of improved pancreatic function.

In certain embodiments, the application provides methods for increasing the production of insulin in a cell composition by culturing the cell composition in the presence of a BMP family member, such as a GDF8/GDF11 subfamily member, or an activator of a BMP signaling pathway. Optionally, the BMP family member has one or more of the following characteristics: an amino acid sequence that is at least 80% identical to a human GDF 11 or GDF8; ability to bind to an ActRIIA and/or ActRIIB receptor; an ability to increase Smad2 and/or Smad3 phosphorylation in a stem cell; an ability to increases expression of a gene that is positively regulated by Smad4. In certain embodiments, the application provides methods for increasing insulin production in a cell composition comprising stem cells, the method comprising contacting the cell composition with a substance that stimulates an ActRIIA and/or ActRIIB signaling pathway. Activators of an ActRIIA and/or ActRIIB signaling pathway may, for example, cause an increase in Smad2 and/or Smad3 function or phosphorylation, or increase expression of a gene that is positively regulated by Smad4. A BMP signaling pathway may be activated by causing overexpression of one or more positive regulators, such as Smad2, Smad3, or a BMP family member. A BMP signaling pathway may also be activated by inhibiting an inhibitor, such as noggin or chordin. Optionally, the cell composition comprises beta cell precursor cells. In certain embodiments, the cell composition is derived from embryonic stem cells that have been cultured in the presence of a retinoid.

In certain embodiments, the application provides a method for promoting the maturation of beta cell precursor cells, the method comprising contacting the beta cell precursor cells with a BMP family member.

In certain aspects the application provides methods for increasing the number of beta cell precursor cells in a cell composition by culturing the cell composition in the presence of an antagonist of a BMP signaling pathway. The antagonist of a BMP signaling pathway may be a secreted polypeptide, such as noggin, chordin or follistatin that binds directly to a BMP family member.

In certain aspects, the application provides cell culture methods that employ sequential inhibition and activation of a BMP signaling pathway to obtain insulin-producing cells. For example, the cells may be cultured in the presence of a noggin, chordin or follistatin and then cultured in the presence of a BMP family member, such as a member of the GDF8/GDF11 subfamily. Optionally, the stem cells are islet precursor cells.

In certain aspects, the application provides methods for ameliorating a condition associated with insufficient pancreatic function by administering a BMP family member, such as a GDF8/GDF 11 subfamily member, or an activator of a BMP signaling pathway. Suitable subjects include those that have been diagnosed with type I or type II diabetes.

In certain aspects, the application provides methods for increasing the production of beta cell precursor cells in a subject, the method comprising administering to the subject a substance that inhibits a BMP signaling pathway, such as a noggin, chordin or follistatin. In some instances, sequential use of BMP pathway antagonists and activators may be desirable.

Insulin production in a subject may also be enhanced by administering a composition comprising insulin producing cells produced according to a method of the application.

In certain aspects, the application provides methods for assessing the effectiveness of a test agent for modulating the development of insulin producing cells. For example, a method may comprise: forming a mixture comprising the test agent and a BMP pathway polypeptide; and detecting binding between the test agent and the polypeptide, wherein a test agent that binds the BMP pathway polypeptide specifically and with an affinity of at least 10⁻⁴M has an increased likelihood of being effective for modulating the development of insulin producing cells. In a further method embodiment, a cell is cultured with the test agent; and an activity of a BMP signaling pathway is detected, wherein a test agent that increases the activity of a BMP signaling pathway has an increased likelihood of being effective for increasing insulin production in a cell. Test agents for promoting formation of beta cell precursor cells may similarly be tested by determining whether they cause a decreases in the activity of a BMP signaling pathway. Cells for use in such assays preferably express an ActRIIA and/or ActRIIB receptor. Optionally, assay cells comprise a reporter gene, wherein expression of the reporter gene is positively or negatively regulated by a BMP signaling pathway. The activity of a BMP signaling pathway may be assessed in a variety of ways, including measuring expression of a reporter gene regulated by a BMP signaling pathway or by measuring a change in one or more components of the BMP signaling pathway. For example, phosphorylation of one or more pathway proteins, such as ActRIIA, ActRIIB, Smad2 and Smad3, may be evaluated.

The embodiments and practices of the present application, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1. Gdf11 expression in the pancreatic islet progenitor cell niche. E13.5 pancreatic cells expressing ngn3 ((blue staining) are detected by antisense RNA in situ hybridization. ngn3⁺cells are found adjacent and contiguous to Gdf11-expressing epithelial cells stained brown with an antiserum specific for Gdf11. Original magnification 16×.

FIG. 2. Defective pancreatic development in embryos deficient for Gdf11. Gdf11⁺indicates that the observed phenotype is indistinguishable in Gdf11^{+/+} and Gdf11^{+/-} embryos. (a,b) Whole mounted preparations of pancreas from Gdf11^{+/-} and Gdf11^{-/-} mice at postnatal day 1. (c) Pancreatic mass unaffected in Gdf11^{-/-} mice. Data are shown as the average measurements from at least 4 mice of indicated genotypes±standard error of the mean. (d,e) Reduced branching of pancreatic epithelium in E13.5 Gdf11^{-/-} mice. Dark epithelium is stained using antiserum specific for PDX1. (f) Quantification of branching defect in Gdf11-deficient mice. Arbitrary units measure individual epithelial clusters with a central lumen. Data are shown as the average measurements from at least 4 mice of indicated genotypes±standard error of the mean. (g,h) Reduced exocrine acinar cell development in Gdf11-deficient mice at E17.5. Dark epithelium is stained using antiserum specific for carboxypeptidase A (carbA). Intralobular septae indicated by the double-headed arrow. These septae are reduced in number in Gdf11-deficient mice. (i) Quantification of morphometry showing significant reduction of exocrine acinar cell volume in Gdf11^{-/-} mice. Original magnification 4×(a,b), 16×(d,e) and 10×(g,h)

FIG. 3. Defective pancreatic islet β-cell and α-cell numbers in Gdf11 deficient mice. (a-c) Immunohistochemical detection of insulin (brown staining) at E17.5 in mice with the indicated genotypes. (d) Quantification of E17.5 β-cell mass by point-counting morphometry in Gdf11^{30 /+}, Gdf11^{+/-}, and Gdf11^{-/-} pancreata. Data are shown as the average measurements from at least 4 mice of indicated genotypes±standard error of the mean. (e-g) Immunohistochemical detection of glucagon (brown staining) at E17.5 in mice with the indicated genotypes. (h-j) Pancreatic islet cell expression of insulin (FITC, green) and glucagon (Cy3, red) at postnatal day 1 (n=3 mice per genotype). Representative samples are shown. (k) Ratio of β-cell to α-cell volume in mice with indicated genotypes. Data are shown as the average measurements±standard error of the mean (n=3 mice per genotype). Original magnification 4×(a-c, e-g) and 63×(h-j).

FIG. 4. Premature expansion and increased numbers of ngn3⁺ pancreatic cells in Gdf11-deficient mice. Pancreatic ngn3 expression (blue staining) detected by antisense RNA in situ hybridization in Gdf11^{+/+} (a-c), Gdf11^{+/-} (d-f) and Gdf11^{-/-} embryos (g-i) at the indicated embryonic stages. (j) Quantification of ngn3⁺ pancreatic cells/mm². Data from 3-4 embryos per genotype are presented as the average±standard error of the mean. Original magnification 10×(a-i).

FIG. 5. Pancreatic defects in E17.5 Smad2 mutant embryos. (a,b) Neurogenin-3 expression (blue staining) detected by antisense RNA in situ hybridization. By E17.5 in wildtype pancreas, only small clusters of ngn3⁺ cells are detected. In Smad2 heterozygous mutants, ngn3 expression is abnormally persistent in clustered periductal epithelial cells. (c,d) Nk×2.2 expression (brown nuclei) adjacent to ductal epithelium. (e,f) Nk×6.1 expression (brown nuclei marked by arrows). (g,h) Insulin expression (green) detected by IHC and confocal microscopy. Inset, h: ngn3⁺ nuclei (Cy3, red) adjacent to insulin⁺ cells (FITC, green). These images are representative of 5 or more animals per genotype. Original magnification 4×(a-f), 25×(g,h).

FIG. 6. Defects of β-cell maturation in Gdf11^{-/-} mice. Measurements from E17.5 pancreata in the indicated genotypes. (a-b) Accumulation of cells expressing Nkx6.1 (darkly stained nuclei) in Gdf11^{-/-} mice. (c) Quantification of Nkx6.1⁺ nuclei per mm² tissue. Data here and in panels f and i are shown as the average measurements ±standard error of the mean (n=3 mice per genotype). (d-e) Accumulation of cells expressing Nk×2.2 (darkly stained nuclei) in Gdf11^{-/-} mice. (f) Quantification of Nk×2.2⁺nuclei per mm² tissue. (g,h) Reduction of Is11⁺ pancreatic βcells in Gdf11−/− mice. (i) Quantification of Is11⁺ nuclei per mm² tissue. Original magnification 16×, (a-b, d-e, g,h).

FIG. 7. Insulin yields from embryoid bodies derived from human embryonic stem cells (UC06) following treatment with the sequence of indicated conditions. Conditions #1-9: exposure of human embryoid bodies to 2 micromolar retinoic acid (RA) for 7 days, followed by exposure for 7 days to (1) two micromolar RA, (2) 10 mM Nicotinamide, (3) 10 micromolar LY294002, (4) Both 10 mM Nicotinamide and 10 micromolar LY294002, (5) 10 ng/mL GDF8, (6) 10 nM Nicotinamide and 10 ng/mL GDF8, (7) 10 mM Nicotinamide and 10 micromolar LY294002 and 10 ng/mL GDF8, (8) 2 nM activin A, or (9) 10 mM Nicotinamide and 2 nM activin A. Conditions #10-18: exposure of human embryoid bodies to 100 nM retinoic acid (RA) for 7 days, followed by exposure for 7 days to (10) 100 nM RA, (11) 10 mM Nicotinamide, (12) 10 micromolar LY294002, (13) Both 10 nM Nicotinamide and 10 micromolar LY294002, (14) 10 ng/mL GDF8, (15) 10 nM Nicotinamide and 10 ng/mL GDF8, (16) 10 mM Nicotinamide and 10 micromolar LY294002 and 10 ng/mL GDF8, (17) 2 nM activin A, (18) 10 mM Nicotinamide and 2 nM activin A. Results are average of triplicate samples.

### DETAILED DESCRIPTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article, unless context clearly indicates otherwise. By way of example, “an element” means one element or more than one element.

An “ActRIIA or ActRIIB signaling pathway” refers to polypeptides and polypeptide interactions that participate in transducing or otherwise effectuating changes in the properties of a cell upon stimulation of an ActRIIA and/or ActRIIB receptor (e.g. by contacting the receptor with a natural ligand such as a GDF8 or GDF11), including the receptors themselves. An example of an ActRIIA or ActRIIB signaling pathway is the ActRII-ActRI-Smad2/3-Smad4 transcriptional regulation pathway.

The term “adult stem cell” is used herein to refer to a stem cell obtained from any non-embryonic tissue. For example, cells derived from fetal tissue and amniotic or placental tissue are included in the term adult stem cell. Cells of these types tend to have properties more similar to cells derived from adult animals than to cells derived from embryonic tissue, and accordingly, for the purposes of this application stem cells may be sorted into two categories: “embryonic” and “adult” (or, equivalently, “non-embryonic”).

“Beta cell precursor cells” are cells having generally mesenchymal, non-cell-cell adherent qualities that form insulin producing cells under appropriate conditions.

A “cell composition” is any composition of matter generated by human manipulation that comprises viable cells as a substantial component. A cell composition may comprise more than one type of viable cell. An “enriched cell composition” is a cell composition comprising a substantially greater purity (i.e. at least twice as pure) of a recognizable cell type than is found in any natural tissue. A “pure cell composition” is a cell composition that comprises at least about 75%, and optionally at least about 85%, 90% or 95% of a recognizable cell type. A recognizable cell type is generally one that has a reasonably uniform morphology, a characteristic set of two or more molecular markers and a functional characteristic. It i s understood that there is likely to be some variation in certain characteristics even within a recognizable cell type. A cell composition may comprise, in addition to cells, essentially any component(s) that are compatible with the intended use for the cell composition. For example, a cell composition may include media, growth factors, pharmaceutically acceptable excipients, preservatives, a solid or semi-solid substrate, a porous matrix or scaffold, nonviable cells or a therapeutic agent.

The term “culturing” includes exposing cells to any condition. While “culturing” cells is often intended to promote growth of one or more cells, “culturing” cells need not promote or result in any cell growth, and the condition may even be lethal to a substantial portion of the cells.

A later cell is “derived” from an earlier cell if the later cell is descended from the earlier cell through one or more cell divisions. Where a cell culture is initiated with one or more initial cells, it may be inferred that cells growing up in the culture, even after one or more changes in culture condition, are derived from the initial cells. A later cell may still be considered derived from an earlier cell even if there has been an intervening genetic manipulation.

A member of the “GDF8/GDF11 subfamily” is a polypeptide (or an encoding nucleic acid) comprising an amino acid sequence that is at least 80% identical to an amino acid sequence of a mature, naturally occurring GDF8 or GDF11 polypeptide, such as the mature human or mouse GDF8 and GDF11 sequences, a fragment thereof. A member of the GDF8/GDF11 subfamily should also retain the ability to stimulate a receptor-mediated signaling pathway.

The term “including” is used herein to mean, and is used interchangeably with, the phrase “including but not limited to”.

The term “islet precursor cell” refers to any cell that has differentiated so as to be recognizably of pancreatic lineage and that differentiates under appropriate conditions to give rise to beta cell precursor cells.

The term “or” is used herein to mean, and is used interchangeably with, the term “and/or”, unless context clearly indicates otherwise.

The term “percent identical” refers to sequence identity between two amino acid sequences or between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Cali., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “stem cell” as used herein refers to an undifferentiated cell which is capable of proliferation and giving rise to at least one more differentiated cell type. “Totipotent stem cells” are stem cells that are capable of giving rise to any cell type of the organism from which the stem cells were obtained. “Pluripotent stem cells” are stem cells that are capable of giving rise to cells of the three major embryonic lineages, the endoderm, mesoderm and ectoderm. “Multipotent stem cells” are stem cells that are capable of giving rise to more than one type of more differentiated cell. The term “stem cell” is also intended to include cells of varying developmental potential that may be obtained by somatic cell nuclear transfer or by causing a differentiated cell to undergo de-differentiation. For the purposes of this disclosure, a stem cell is named by the tissue from which it was obtained. For example, a “neural stem cell” is a stem cell obtained from a neural tissue (or a fluid, such as cerebrospinal fluid that is in contact with neural tissue), a “neuroendocrine stem cell” is a stem cell derived from a neuroendocrine tissue, such as the adrenal gland or the pituitary gland, but specifically excluding the pancreas. An “embryonic stem cell” is a stem cell obtained from an embryo. Many “tissues” are complex and actually contain several different stem cell types. For example, the skin may be considered a tissue, but skin contains neural stem cells of the peripheral nervous system, skin stem cells from the dermis, and stem cells from the blood circulating through the skin. Accordingly, in determining the classification of a stem cell, the true origin, including sub-tissue structures, should be carefully considered.

A “stem cell line” is an enriched or pure cell composition comprising a recognizably distinct stem cell type that, when cultured in appropriate conditions, self-propagates.

2. Methods for Promoting Formation of Beta Cell Precursors and Increasing Insulin Production

In certain aspects the application provides methods for increasing insulin-production in a suitable cell composition by culturing the cell composition in the presence of a BMP family member or other activator of a BMP signaling pathway. Suitable cell populations will generally include any cell populations containing stem cells that retain the ability to develop into insulin-producing cells. As used herein, the term “stem cell” includes cells that are islet cell or beta cell precursors. Preferably a suitable cell population comprises a cell that has undergone an epithelial to mesenchymal cell type shift, but has not undergone a reorganization back to the epithelial cell traits that are characteristic of mature pancreatic beta cells.

While not wishing to be bound to theory, it is contemplated that the development of pancreatic islets involves a shifting of cell characteristics from epithelial to mesenchymal and then back to epithelial again. At an early stage of pancreatic development, the cells are organized in a pancreatic duct. The cells of the pancreatic duct have epithelial characteristics, meaning that the cells adhere closely, possibly through tight junctions, to form sheets. To form islets, cells of the duct become mesenchymal in character, meaning that the adhesions to neighboring cells dissolve and the cells move away from the two dimensional sheet. Then the cells develop adhesive, epithelial qualities again and form into islets, separated from the original ductal cell sheet. In addition, the final steps of islet cell maturation are marked by a cessation of proliferation. Members of the BMP family may encourage the formation of epithelial structures, thereby directing the dispersed mesenchymal cells (e.g. beta cell precursors) to form multicellular islet structures. Members of the BMP family also inhibit proliferation, in part by causing expression of inhibitors of cyclin-dependent kinases (cdks), such as p15, p17, p18, p21, p27, or p57. Inhibitors of the BMP signaling pathway may stimulate the formation of beta cell precursor cells in two ways. First, such inhibitors may stimulate epithelial cells of the duct to adopt mesenchymal characteristics, and second, such inhibitors may prevent formation of the islet epithelial structures.

Without regard to theories of pancreatic development, the present application demonstrates that the development of islet cells and various precursors can be manipulating by altering BMP signaling.

A “BMP family member” may be selected from the naturally occurring members of the TGF-beta/BMP family, and may also be a functional analog thereof. Naturally occurring members of this family are characterized by common sequence homology and as being proteins that are translated as preproprotein precursors. A preprotein precursor contains an N-terminal signal peptide, a prodomains and a mature portion. The mature portion contains six to nine conserved cysteine residues that participate in forming intermolecular disulfide bonds, although a few members (e.g. GDF-9, BMP-15, GDF-3, lefty-1 and lefty-2) contain a serine substitution for a cysteine involved in disulfide bond formation. A dendrogram presented in Chang et al. (2002) Endocrine Rev. 23(6):787-823 lists a number of vertebrate BMP family members, including the following: TGF-beta2, TGF-beta3, TF-betal, GDF-15, GDF-9, BMP-15, BMP-16, BMP-3, GDF-10, BMP-9, BMP-10, GDF-6, GDF-5, GDF-7, BMP-5, BMP-6, BMP-7, BMP-8, BMP-2, BMP-4, GDF-3, GDF-1, GDF 11, GDF8, Activins betaC, betaE, betaA and betaB, BMP-14, GDF-14, MIS, Inhibin alpha, Lefty1, Lefty2, GDNF, Neurteurin, Persephin and Artemin. The term “BMP family member” is intended to include variant forms of any of the naturally occurring polypeptides, including fusion proteins, truncated proteins, and mutant forms, although only if such variants retain the ability to stimulate a receptor-mediated signaling pathway and further have sufficient sequence similarity to a naturally occurring family member as to be recognizable using a sequence comparison algorithm such as BLAST or PILEUP. The term “BMP family member” is also intended to refer both to single polypeptides and homo- and hetero-dimers or other multimeric forms, as well as nucleic acids encoding polypeptides that are BMP family members. Several subgroupings are also identifiable, including the “GDF8/GDF11 subfamily” (e.g. GDF8, GDF11), the “Activin C subfamily” (e.g. Activins betaC, betaE, and BMP-14), the “Activin A subfamily” (e.g. Activin betaA, betab), the “TGF-beta subfamily” (e.g. TGF-beta2, TGF-beta3, TF-beta1), the “BMP-5 subfamily” (e.g. BMP-5, BMP-6, BMP-7), the “BMP-8 subfamily” (e.g. BMP-8), the “GDF-6 subfamily” (e.g. GDF-5, GDF-6, GDF-7), the “BMP-9 subfamily” (e.g. BMP-9, BMP-10), the “BMP-3 subfamily” (e.g. BMP-3, GDF-10), the “GDF-9 subfamily” (e.g. GDF-9, BMP-15) and the “GDF-15 subfamily” (e.g. GDF-15).

A BMP family member may be replaced by, or used in conjunction with, a BMP signaling pathway activator. A BMP signaling pathway activator is a compound that stimulates one or more of the polypeptides or interactions that participate in transducing or otherwise effectuating changes in the properties o f a cell in response to a BMP family member. A BMP signaling pathway includes BMP family members themselves. An example of a BMP signaling pathway is the GDF11-ActRII-ActRI-Smad2/3-Smad4 transcriptional regulation pathway. The BMP family member binds to the extracellular ligand binding domain portion of the ActRII receptor and then forms a complex with ActRI, leading to the inhibition of the Smad7 negative regulator and phosphorylation of the Smad2/Smad3 complex. The Smad2/Smad3 complex associates with Smad4 to regulate expression of certain genes. In certain embodiments, activation of a BMP signaling pathway leads to activation of small inhibitors of cyclin-dependent kinases (cdks), such as p15, p17, p18, p21, p27 and p57. A BMP signaling pathway activator is also a compound that antagonizes an inhibitor of a BMP signaling pathway, such as an anti-noggin or anti-chordin antibody.

Accordingly, certain method embodiments of the application comprise culturing stem cells, preferably embryonic stem cells, to a desired stage and then contacting the cells with a BMP family member, preferably a GDF8/GDF11 subfamily member. Public database references for preferred GDF8/GDF11 are set forth in Table 1, and such references provide guidance as to the mature and pro-polypeptide sequences. The database entries listed in Table 1 are incorporated herein by reference, in their entirety.

In an exemplary embodiments, human embryonic stem cells are cultured in the presence of a retinoid compound, such as all-trans retinoic acid and then cultured in the presence of a GDF8/GDF11 subfamily member, thereby generating a cell composition comprising insulin-producing cells.

TABLE 1
Selected GDF8/GDF11 Subfamily Members

| | | Polypeptide | Nucleic Acid |
|---|---|---|---|
| Protein Name | Organism | Database Ref. No. | Database Ref. No. |
| GDF11 | Human | O95390 | NM_005811 |
| GDF11 | Mouse | Q9Z1W4 | XM_125935 |
| GDF8 | Human | O14793 | AF019627 |
| GDF8 | Mouse | O08689 | U84005 |

In certain embodiments, the application provides methods for increasing the number of beta cell precursor cells in a cell composition by culturing cells in the presence of an inhibitor of a BMP pathway. Inhibitors of a BMP pathway include noggins, chordins, follistatins, twisted gastrulation (TSG), Dan, Cerberus and Xenopus nodal related 3 (Xnr3), to name only a few. In addition, antibodies that bind to BMP family members and prevent receptor activation may also be used as BMP pathway inhibitors. Public database references for preferred BMP pathway inhibitors are set forth in Table 2, and such references provide guidance as to the mature and pro-polypeptide sequences. The database entries listed in Table 2 are incorporated herein by reference, in their entirety.

TABLE 2
Selected Inhibitors of BMP Signaling

| | | Polypeptide | Nucleic Acid |
|---|---|---|---|
| Protein Name | Organism | Database Ref. No. | Ref. No. |
| Noggin | Human | Q13253 | BC034027 |
| Noggin | Mouse | P97466 | NM_008711 |
| Chordin | Human | Q9H2X0 | XM_209529 |
| Chordin | Mouse | NP_034023 | NM_009893 |
| Follistatin | Human | P19883 | M19480 |
| | | | M19481 |
| Follistatin | Mouse | S45321 | X83377 |

In certain embodiments, a cell composition comprising stem cells may be cultured sequentially with an inhibitor of BMP signaling and then with an activator of BMP signaling, thereby obtaining insulin-producing cells.

Stem cells for use in the methods disclosed herein may be essentially any stem cell that has not lost the potential to become a pancreatic hormone-producing cell. The term “stem cell” as used herein refers to an undifferentiated cell which is capable of proliferation and giving rise to at least one more differentiated cell type. Stem cells may be totipotent, pluripotent stem cells or multipotent. Stem cells may also be obtained by somatic cell nuclear transfer or by causing a differentiated cell to undergo de-differentiation. In certain embodiments, stem cells for use with the disclosed methods may be impure, such as stem cells nested in a tissue or in a suspension obtained from a tissue sample. It is now widely believed that most adult tissues include small populations of stem cells, as that term is used herein. Stem cells may also be enriched from tissue samples, and may optionally be purified stem cells. Stem cells may also be used from stem cell lines, and preferably from well-characterized and established stem cell lines. Tissue may be embryonic or “adult” as the term is used herein, including fetal, infant, child and mature animal tissue. Cells need not be obtained from a tissue, and other cell-containing sources that are not generally considered “tissues” may be employed (e.g. cerebrospinal fluid and mucus or secreted fluids of the lung or gut).

In certain embodiments a stem cell for use in a disclosed method is an embryonic stem cell. Examples of mouse embryonic stem cells include: the JM1 ES cell line described in M. Qiu et al., Genes Dev 9, 2523 (1995), and the ROSA line described in G. Friedrich, P. Soriano, Genes Dev 5, 1513 (1991), and mouse ES cells described in U.S. Pat. No. 6,190,910. Many other mouse ES lines are available from Jackson Laboratories (Bar Harbor, Maine). Examples of human embryonic stem cells include those available through the following suppliers: Arcos Bioscience, Inc., Foster City, Cali., CyThera, Inc., San Diego, Cali., BresaGen, Inc., Athens, Ga., ES Cell International, Melbourne, Australia, Geron Corporation, Menlo Park, Calif., Göteborg University, Göteborg, Sweden, Karolinska Institute, Stockholm, Sweden, Maria Biotech Co. Ltd.—Maria Infertility Hospital Medical Institute, Seoul, Korea, MizMedi Hospital—Seoul National University, Seoul, Korea, National Centre for Biological Sciences/Tata Institute of Fundamental Research, Bangalore, India, Pochon CHA University, Seoul, Korea, Reliance Life Sciences, Mumbai, India, Technion University, Haifa, Israel, University of California, San Francisco, Calif., and Wisconsin Alumni Research Foundation, Madison, Wis. In addition, examples of embryonic stem cells are described in the following U.S. patents and published patent applications Nos.: 6,245,566; 6,200,806; 6,090,622; 6,331,406; 6,090,622; 5,843,780; 20020045259; 20020068045. In preferred embodiments, the human ES cells are selected from the list of approved cell lines provided by the National Institutes of Health and accessible at http://escr.nih.gov. In certain preferred embodiments, an embryonic stem cell line is selected from the group consisting of: the WA09 line obtained from Dr. J. Thomson (Univ. of Wisconsin) and the UC01 and UC06 lines, both on the current NIH registry.

In certain embodiments, a stem cell for use in disclosed methods is a stem cell of neural or neuroendocrine origin, such as a stem cell from the central nervous system (see, for example U.S. Pat. Nos. 6,468,794; 6,040,180; 5,753,506; 5,766,948), neural crest (see, for example, U.S. Pat. Nos. 5,589,376; 5,824,489), the olfactory bulb or peripheral neural tissues (see, for example, Published US Patent Applications 20030003574; 20020123143; 20020016002 and Gritti et al. 2002 J Neurosci 22(2):437-45), the spinal cord (see, for example, U.S. Pat. Nos. 6,361,996, 5,851,832) or a neuroendocrine lineage, such as the adrenal gland, pituitary gland or certain portions of the gut (see, for example, U.S. Pat. Nos. 6,171,610 and PC12 cells as described in Kimura et al. 1994 J. Biol. Chem. 269: 18961-67). In preferred embodiments, a neural stem cell is obtained from a peripheral tissue or an easily healed tissue from a patient in need of cells that produce a pancreatic hormone, thereby providing an autologous population of cells for transplant.

In certain embodiments, hematopoietic or mesenchymal stem cells may be employed in a disclosed method. Recent studies suggest that marrow-derived hematopoietic (HSCs) and mesenchymal stem cells (MSCs), which are readily isolated, have a broader differentiation potential than previously recognized. Purified HSCs not only give rise to all cells in blood, but can also develop into cells normally derived from endoderm, like hepatocytes (Krause et al., 2001, Cell 105: 369-77; Lagasse et al., 2000 Nat Med 6: 1229-34). MSCs appear to be similarly multipotent, producing progeny that can, for example, express neural cell markers (Pittenger et al., 1999 Science 284: 143-7; Zhao et al., 2002 Exp Neurol 174: 11-20). Examples of hematopoietic stem cells include those described in U.S. Pat. Nos. 4,714,680; 5,061,620; 5,437,994; 5,914,108; 5,925,567; 5,763,197; 5,750,397; 5,716,827; 5,643,741; 5,061,620. Examples of mesenchymal stem cells include those described in U.S. Pat. Nos. 5,486,359; 5,827,735; 5,942,225; 5,972,703, those described in PCT publication nos. WO 00/53795; WO 00/02654; WO 98/20907, and those described in Pittenger et al. and Zhao et al., supra.

Stem cell lines are preferably derived from mammals, such as rodents (e.g. mouse or rat), primates (e.g. monkeys, chimpanzees or humans), pigs, and ruminants (e.g. cows, sheep and goats), and particularly from humans. In certain embodiments, stem cells are derived from an autologous source or an HLA-type matched source. For example, stem cells may be obtained from a subject in need of pancreatic hormone-producing cells (e.g. diabetic patients in need of insulin-producing cells) and cultured by a method described herein to generate autologous insulin-producing cells. Other sources of stem cells are easily obtained from a subject, such as stem cells from muscle tissue, stem cells from skin (dermis or epidermis) and stem cells from fat. Insulin-producing cells may also be derived from banked stem cell sources, such as banked amniotic epithelial stem cells or banked umbilical cord blood cells.

In certain embodiments, a stem cell may be derived from a cell fusion or dedifferentiation process, such as described in the following US patent application: 20020090722, and in the following PCT applications: WO200238741, WO0151611, WO9963061, WO9607732.

In some preferred embodiments, a stem cell line should be compliant with good tissue practice guidelines set for the by the U.S. Food and Drug Administration (FDA) or equivalent regulatory agency in another country. Methods to develop such a cell line may include donor testing, and avoidance of exposure to non-human cells and products during derivation of the stem cell lines. Preferably the stem cell line can be prepared and used without the use of a feeder layer or any type of virus or viral vector.

In certain preferred embodiments, both the stem cells and differentiated cells of the methods and compositions disclosed herein have a wild-type genotype, meaning that the genotype of the cells is a genotype that may be found in a subject organism naturally. For example, cells having chromosomal rearragements as a result of culture treatments are not cells having a wild-type genotype. As a further example, cells that have been transfected with an integrating nucleic acid construct will not (except in cases of perfect excision) have a wild-type genotype. The term “genotype” does not refer to peripheral modifications to the genomic nucleic acids, such as methylation, and therefore, cells having a naturally occurring genetic makeup may have unnatural phenotypes as an effect of changes in methylation or other modifications.

Certain embodiments of the methods disclosed herein are advantageous in part because they permit the generation of insulin-producing cell compositions from starting materials, such as certain stem cell lines, that are available, as a practical matter, in sufficient quantities for formation of a therapeutically effective insulin-producing implant. By contrast, for example, fetal pancreatic tissue, and particularly human fetal pancreatic tissue, is only available in small quantities, making it difficult or impossible to assemble sufficient material to form a therapeutically effective implant.

In certain embodiments, an insulin-producing cell is exposed to an additional culture condition. For example, insulin-producing cells may be treated with any of the various agents, and functional analogs thereof, that are known to stimulate insulin production or beta-cell proliferation. Examples of such agents include IGF-1 (e.g. at a concentration of 10 ng/ml), glucagon-like peptides (e.g. GLP-1), exendin-4, HGF, and reagents that increase cAMP levels, such as membrane permeable forms of cAMP and forskolin.

3. Insulin-Producing Cell Compositions

In certain embodiments, the application provides insulin-producing cell compositions produced according to any of the methods disclosed herein. Insulin-producing cell compositions may be in any form, including, preferably, in insulin-producing cell clusters, but optionally in dispersed cell suspensions, confluent cell cultures, seeded on a matrix or other cell support, etc.

In further embodiments, the invention relates to insulin-producing cell compositions in which at least about 50% of the cells are positive for insulin production, optionally at least 75% of the cells are positive for insulin production and preferably at least 85%, 90% or 95% of the cells are positive for insulin production. In certain embodiments, most of the cells, and preferably greater than 80%, 90% or 95% of the cells, that produce insulin are negative for other pancreatic hormones that are not naturally produced by native pancreatic insulin producing cells, such as glucagon.

In certain embodiments, an insulin-producing cell composition comprises at least about 1000 nanograms (ng) of insulin per milligram of total protein, optionally at least about 5000 nanograms of insulin per milligram of total protein and preferably at least about 10000 nanograms of insulin per milligram of total protein. In embodiments where the insulin-producing cell composition comprises islet-like cell clusters of roughly 300-400 μm in diameter, the clusters produce greater than 0.2 ng of insulin per hour, and preferably greater than 0.5 ng of insulin per hour. In preferred embodiments, insulin production by the insulin-producing cell composition is stimulated by exposure to glucose.

In certain embodiments, insulin-producing cell compositions comprise cells that are positive for one or more of the following markers: insulin (any of the various chains), islet-1, PDX1, GLUT2, glucokinase, a cdk inhibitor, and a tight junction protein, such as a connexin. In certain embodiments, at least about 50% of the cells in an insulin-producing cell composition are not proliferative. Proliferating cells may be detected by a variety of ways known in the art, including staining with Ki67, a nuclear marker of proliferating cells, or incorporation of labeled nucleotide (e.g. tritiated thymidine or bromodeoxyuridine). In preferred embodiments, insulin-producing cell compositions do not form neoplastic growths when implanted in a subject. It is understood that biological systems are tremendously variable and, depending on host and implant characteristics, even a very safe insulin-producing cell composition is likely to form, or appear to form, a neoplastic growth at some low frequency. In certain embodiments the insulin-producing cell compositions of the invention produce a neoplastic growth in a fewer than 30% of implanted subject, optionally in fewer than 1% of implanted subjects and preferably in fewer than 0.1% of implanted subjects.

4. Administration of Insulin-Producing Cell Compositions and/or Factors

In additional embodiments, the application provides methods for ameliorating, in a subject, a condition related to insufficient pancreatic function by administering to the subject an effective amount of an insulin producing cell composition. In certain embodiments, administration of a BMP signaling activator may be used to ameliorate a condition related to insufficient pancreatic function. In yet another embodiments, administration of a BMP signaling inhibitor may be used to ameliorate a condition related to insufficient pancreatic function. The benefit from an inhibitor versus an activator of BMP signaling may depend on whether regeneration of insulin-producing cells is limited by a failure in maturation of beta cell precursor cells, or a failure in the formation of beta cell precursor cells. Optionally, a subject may be administered a BMP signaling inhibitor followed by an activator. BMP signaling inhibitors or activators may be coadministered with insulin-producing cells. Preferrably, a sufficient amount of one or more of the above therapeutic compositions is administered to a subject to cause an increase in blood insulin levels or an improvement in glucose homeostasis. Glucose homeostasis may be tested by administering a dose of glucose and monitoring the kinetics with which blood glucose levels decline. Conditions related to insufficient pancreatic function include the various forms of diabetes mellitus (e.g. type I and type II), NOD mice (a type I diabetes model), the streptozotocin-induced diabetes rodent model, surgically-induced diabetes models and diseases resulting from dysfunctional islet growth (e.g. insulinomas). Administration of an insulin-producing cell composition may not produce a permanent ameliorating effect, and periodic dosing, such as on a weekly, monthly or yearly basis may be beneficial.

In preferred embodiments, an effective dose of insulin-producing cell composition comprises administering at least about one islet-like cell cluster of the invention (or an equivalent number of cells) per islet that is naturally present in the subject organism. For example, mice have about 300-500 islets, rats have about 3000-5000 islets and humans have about 1,000,000 islets, and accordingly, a preferred dosage is about 300-500 islet-like cell clusters for a mouse, about 3000-5000 islet-like cell clusters for a rat and about 1,000,000 islet-like cell clusters for a human. The number of islets per organism is proportional to average body mass (20-30 grams, mouse, 200-300 grams, rat, 60-70 kilograms, human) and it may be desirable to administer a dosage that is proportional to body mass of the subject. In instances when an islet-like cell cluster is less efficient at producing insulin than a native islet, or where an insulin-producing cell composition is subject to cell mortality (e.g. in the case of host immune system-mediated rejection), the dosage may be increased proportionally.

In certain embodiments, the invention relates to therapeutic compositions comprising insulin-producing cell compositions and methods for making such therapeutic compositions. Therapeutic compositions include an insulin-producing cell compositions disclosed herein and/or made by the methods disclosed herein, as well as mixtures comprising such insulin-producing cell compositions and a therapeutic excipient. Examples of therapeutic excipients include matrices, scaffolds or other substrates to which cells may attach (optionally formed as solid or hollow beads, tubes, or membranes), as well as reagents that are useful in facilitating administration (e.g. buffers and salts), preserving the cells (e.g. chelators such as sorbates, EDTA, EGTA, or quaternary amines or other antibiotics), or promoting engraftment.

Cells may be encapsulated in a membrane to avoid immune rejection. By manipulation of the membrane permeability, so as to allow free diffusion of glucose and insulin back and forth through the membrane, yet block passage of antibodies and lymphocytes, normoglycemia may be maintained (Sullivan et al. (1991) Science 252:718). In a second approach, hollow fibers containing cells may be immobilized in a polysaccharide alginate. (Lacey et al. (1991) Science 254:1782). Cells may be placed in microcapsules composed of alginate or polyacrylates. (Lim et al. (1980) Science 210:908; O'Shea et al. (1984) Biochim. Biochys. Acta. 840:133; Sugamori et al. (1989) Trans. Aim. Soc. Artif. Intern. Organs 35:791; Levesque et al. (1992) Endocrinology 130:644; and Lim et al. (1992) Transplantation 53:1180).

Additional methods for encapsulating cells are known in the art. (Aebischer et al. U.S. Pat. No. 4,892,538; Aebischer et al. U.S. Pat. No. 5,106,627; Hoffman et al. (1990) Expt. Neurobiol. 110:39-44; Jaeger et al. (1990) Prog. Brain Res. 82:41-46; and Aebischer et al. (1991) J. Biomech. Eng. 113:178-183, U.S. Pat. No. 4,391,909; U.S. Pat. No. 4,353,888; Sugamori et al. (1989) Trans. Am. Artif. Intern. Organs 35:791-799; Sefton et al. (1987) Biotehnol. Bioeng. 29:1135-1143; and Aebischer et al. (1991) Biomaterials 12:50-55).

The site of implantation of insulin-producing cell compositions may be selected by one of skill in the art. In general, such as site preferably has adequate blood perfusion to allow the cells to sense blood conditions and secrete hormones and other factors into the general circulation. Exemplary implantation sites include the liver (via portal vein injection), the peritoneal cavity, the kidney capsule and the pancreas.

6. Methods for Identifying Agents for Modulation of Pancreatic Cell Development

In certain aspects, the application provides methods for assessing the effectiveness of a test agent for modulating the development of insulin producing cells. Such methods may be used to screen libraries of test agents. In general, a method of this type involves assessing the ability of the test agent to interfere with or promote BMP pathway signaling. Two non-limiting categories of assays include biochemical assays and cell-based assays.

In certain embodiments, a test agent may be mixed with a BMP pathway polypeptide; and a test agent that binds the BMP pathway polypeptide specifically and with an affinity of at least 10⁻⁴ M, and preferably 10⁻⁵, 10⁻⁶, 10⁻⁷ or less, has an increased likelihood of being effective for modulating the development of insulin producing cells. An agent that binds may inhibit signaling, as in the case of an agent that binds and titrates a way a BMP family member. Alternatively, an agent that binds may activate, as in the case of an agent that binds to an ActRIIA or ActRIIB receptor and mimics activation by a BMP family member. Test agents may be assessed in an assay system that measures binding between a BMP family member, such as a GDF8/GDF11 family member, and a receptor, such as an ActRIIA or ActRIIB.

In a further method embodiment, a cell is cultured with the test agent; and an activity of a BMP signaling pathway is detected, wherein a test agent that increases the activity of a BMP signaling pathway has an increased likelihood of being effective for increasing insulin production in a cell. Test agents for promoting formation of beta cell precursor cells may similarly be tested by determining whether they cause a decreases in the activity of a BMP signaling pathway. Cells for use in such assays preferably express an ActRIIA and/or ActRIIB receptor. Optionally, assay cells comprise a reporter gene, wherein expression of the reporter gene is positively or negatively regulated by a BMP signaling pathway. A reporter gene may be designed to be activated by Smad4, which is positively regulated by BMP signaling. Examples of Smad4 regulated genes include cdk inhibitors, such as p15, p17, p18, p21, p27 and p57. The activity of a BMP signaling pathway may also be assessed by measuring a change in one or more components of the BMP signaling pathway. For example, phosphorylation of one or more pathway proteins, such as ActRIIA, ActRIIB, Smad2 and Smad3, may be evaluated.

7. Methods for Assessing Candidate Islet Cell Differentiation Factors and Other Test Compounds

In certain embodiments, the application provides methods for obtaining beta cell precursor cell populations as well as insulin-producing cells, and such cells may be used for a variety of purposes, such as the identification of markers for these cell types.

In certain aspects the application provides methods for assessing whether a test agent has beta cell precursor cell differentiation activity. An exemplary embodiment of such a method may comprise contacting beta cell precursor cells with a test agent and detecting a beta cell marker. Generally, a test agent that stimulates the formation of cells expressing islet cell markers has beta cell precursor cell differentiation activity activity. The term “beta cell marker” is intended to include any phenotype that is distinctive of one or more islet cell types, including various protein, nucleic acid, morphological, biochemical (e.g. metabolic or transport) or other phenotypes. Examples of beta cell markers include the following polypeptides or the corresponding RNA transcript: insulin (any of the various chains, including, for example, C-peptide, mature insulin or proinsulin), GLUT2, glucokinase, PDX-1, IAPP, SUR1, PC1/3, PC2, KIR6.2, pancreatic polypeptide, somatostatin, glucagon, glucokinase and C-peptide. In an illustrative embodiment, the subject cells can be used to screen various compounds or natural products, such as small molecules or growth factors. The efficacy of the test agent can be assessed by generating dose response curves. A control assay can also be performed to provide a baseline for comparison.

In certain embodiments, methods of the application relate to the identification of pancreatic developmental markers. For example, expression patterns of established markers may be monitored at one or more stages of differentiation of stem cells into beta cell precursors and insulin-producing cells. Markers may be assessed using standard methods, including Northern blotting, RT-PCR, in situ hybridization (ISH), immunohistochemistry (IHC) as well as nucleic acid or protein array or microarray-based methods. In certain embodiments, monitoring production of one or more gene products will be useful to identify candidate cell-surface proteins for FACS-based purification strategies for insulin-producing cell precursors.

In certain embodiments, the application provides methods for identifying affinity reagent that bind to cells at various stages of pancreatic development. Affinity reagents include antibodies, and preferably monoclonal antibodies, targeting peptides (e.g. peptides selected from a high diversity phage display library), RNA or DNA aptamers. The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic expressing and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody includes polyclonal, monoclonal, or other purified preparations of antibodies and recombinant antibodies. In certain embodiments, beta cell precursors or insulin producing cells may be used to screen a plurality of affinity reagents. The cells themselves may be used for the screening, or membrane or protein extracts may be used. Likewise, cell surface proteins may be selectively labeled and used to screen a plurality of affinity reagents. In a preferred embodiment, the plurality of affinity reagents to be screened is a library of monoclonal antibodies. An affinity reagent detected as binding to a cell such as an beta cell precursor cell may be tested on tissue samples for capability to detect particular subpopulations of pancreatic or pre-pancreatic cells, and it is of particular interest to identify affinity reagents that are useful in the identification of natural populations of cells that are precursors of beta cells or other islet cells.

Yet another aspect of the present application provides methods for screening various compounds for their ability to modulate insulin-producing cells, such as, for example, by affecting growth, proliferation, maturation or differentiation, or by affecting insulin production, secretion or storage, as well as compounds that may improve graft performance (e.g. result in a longer-lasting graft, improved insulin production, or changes in proteins that interact with the host immune system). In an illustrative embodiment, the subject cells can be used to screen various compounds or natural products, such as small molecules or growth factors. Such compounds may be tested for essentially any effect, with exemplary effects being cell proliferation or differentiation, insulin production, or cell death. In further embodiments, insulin-producing cells may used to test the activity of compounds/factors to promote survival and maturation, and further, since certain cells produced according to methods disclosed herein have one or more properties of islet cells, specifically β-cells, such cells may be used to identify factors (or genes) that regulate production, processing, storage, secretion, and degradation of insulin or other relevant proteins (like IAPP, glucagon, including pro-glucagon, GLPs, etc) produced in pancreatic islets. In further embodiments, an insulin-producing cell may be modified, such as by genetic modification, to become hyperproliferative. Such hyperproliferative cells may be contacted with compounds to identify, for example, anti-proliferative and anti-neoplastic agents (e.g. agents that inhibit cell growth or promote cell death). The efficacy of the compound can be assessed by generating dose response curves from data obtained using various concentrations of the compound. A control assay can also be performed to provide a baseline for comparison. Identification of the progenitor cell population(s) amplified in response to a given test agent can be carried out according to such phenotyping as described above. Assays such as those described above may be carried out in vitro (e.g. with cells in culture) or in vivo (e.g. with cell implanted in a subject).

8. Methods for Identifying Stem Cells

In certain embodiments, the application relates to methods for identifying a cell that has the potential to develop into a pancreatic cell, and particularly an insulin-producing cell. In one aspect, the method comprises providing a stem cell line, or other multipotent cell line, and differentiating the cell line so as to obtain an insulin-producing cell composition. At the beginning of the differentiation process, or at some stage within the differentiation process, the differentiating cells are mixed with a cell of interest. The differentiation of the cell of interest may then be assessed. A cell of interest that is able to differentiate into an insulin-producing cell is a cell that has the potential to develop into an insulin-producing cell. In further embodiments, the cell may be assessed for the production of other pancreatic products, such as glucagons, to identify cells that have the potential to develop into other types of pancreatic cells. In certain embodiments, a pancreatic tissue (e.g. ductal tissue, adult pancreatic tissue, fetal pancreatic tissue, etc.) may be dissociated into a cell suspension, and clumps of cells or single cells are used as the cell of interest in the above method embodiments, thereby permitting a rapid screen of pancreatic cells for candidate pancreatic progenitors. By using inhibitors of BMP signaling, it will be possible to obtain large numbers of beta cell precursor cells, which enables identification of markers for this population of cells.

In one embodiment, insulin-producing cell compositions and methods for generating such compositions may be used to assess the developmental potential of a cell of interest. In some embodiments, the developmental potential of a cell of interest may be determined by mixing the cell of interest with cells during the process of making beta precursor or insulin-producing cells (i.e. co-culturing). The cell of interest is then tracked (for example by a transgenic marker) to determine the types of cells that arise from it. In an exemplary embodiment, the cell of interest is mixed with differentiating neural or neuroendocrine stem cells.

In certain embodiments, culture systems for making insulin-producing cell compositions may be used as part of an assay to identify candidate pancreatic endocrine precursor cells. Current evidence suggests that such precursors exist as single cells or small cell clusters within or closely associated with pancreatic epithelium. In certain embodiments, cell compositions in the process of differentiating into beta cell precursor cells or insulin-producing cells provide the appropriate cellular microenvironment to permit pancreas-derived endoderm to integrate and differentiate. In certain embodiments, cells of a pancreatic tissue are fractionated and mixed, either as populations of cells or as single cells, into cells being differentiated into insulin-producing cell compositions. Cells of the pancreatic tissue that develop into insulin-producing cells are candidate pancreatic stem cells. In certain embodiments, instead of a co-culture, a fraction of cells that are in the process of differentiating into insulin-producing cell compositions may be used in the culture medium of the cells of interest. Fractions that may be used include conditioned media or other preparations of secreted material, extracellular matrix, membrane preparations, total soluble protein, soluble cellular protein and other portions of cells that are in the process of differentiating into beta cell precursors or insulin-producing cells.

### EXAMPLES

The invention now being generally described, it will be more readily understood by reference to the following examples, which are included merely for purposes of illustration of certain aspects and embodiments of the present invention, and are not intended to limit the invention.

### Example 1

### GDF-11 and Pancreatic Development

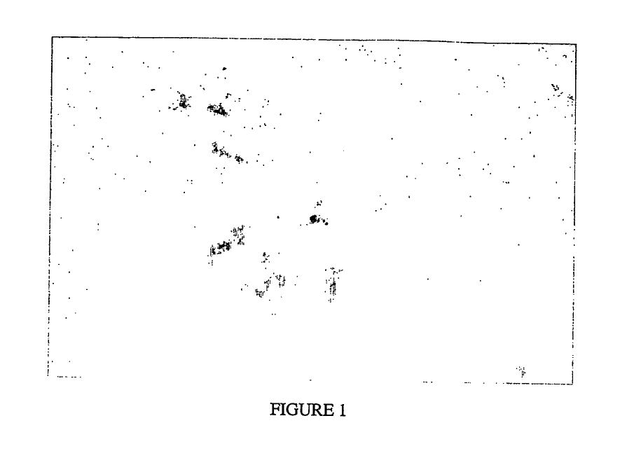

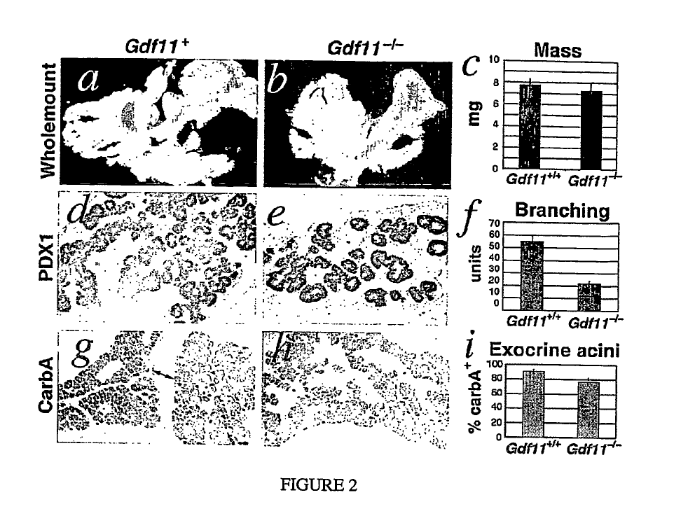

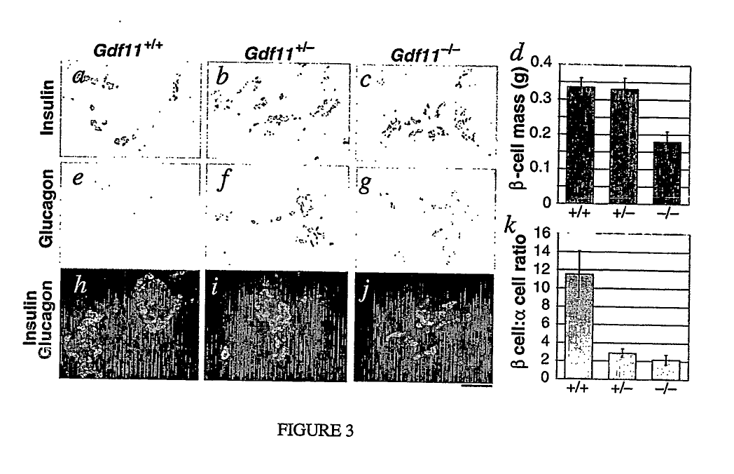

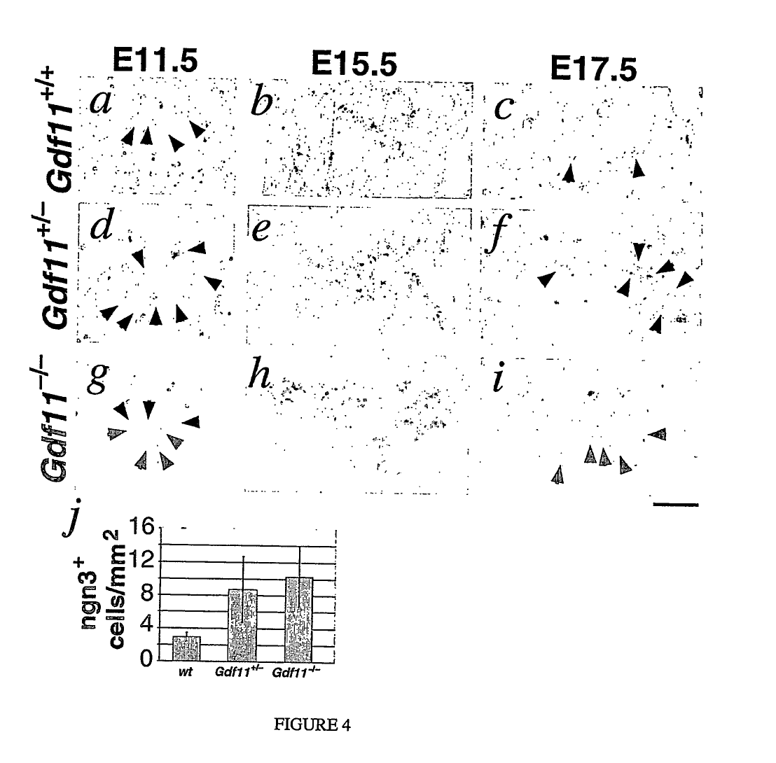

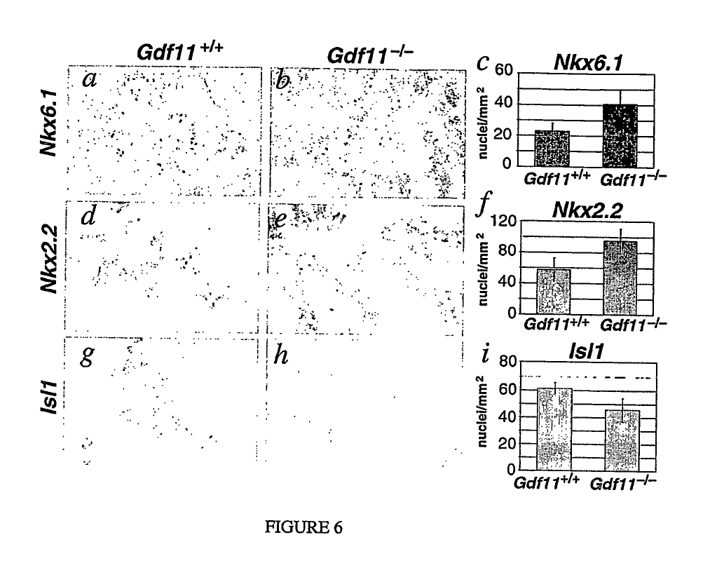

Growth and differentiation factor 11 (GDF-11; also known as GDF11) is a recently identified member of the TGF-βligand superfamily (Nakashima et al., 1999; Gamer et al., 1999). Homozygous null GDF-11 mutants manifest several defects including skeletal transformations, and abnormal Hox gene expression that resemble defects observed in ActRIIB mutants (McPherron and Lee, 1999). GDF-11 expression in pancreatic epithelium begins by E9.5-10 in pancreatic epithelium, continues there throughout gestation, and is later abundantly expressed in isolated adult islets. We have characterized foregut development in GDF-11 mutant embryos, and found (1) hyposplenism with splenic malformations, (2) defects in axial patterning of stomach epithelium and mesenchyme, and (3) severe pancreatic defects including islet hypoplasia, defects in islet cell differentiation, increased accumulation of neurogenin3-expressing cells (presumptive islet cell precursors) during embryonic pancreas formation, and reduced β-cell mass. Thus, morphogenetic defects in pancreas development, are strikingly similar in GDF-11 and ActRIIB mutants (Kim et al., 2000). Our analysis identifies GDF-11 as a candidate TGF-β ligand that regulates development of pancreatic islet precursor cells and subsequent maturation of pancreatic β-cells to functioning insulin-producing and secreting cells. Data are shown in FIGS. 1-4 and 6.

### Example 2

### Smad2 and Pancreatic Development

Numerous in vitro and in vivo studies demonstrate that Smad2 activity is regulated both by ActRIIA and ActRIIB. Smad2 encodes a transcription factor which is phosphorylated by type I activin or TGF-β receptors after ligand binding to type II and type I receptors. In mice, Smad2, ActRIIA, ActRIIB and other TGF-β signaling components are expressed in embryonic pancreas, and later in adult islets. We investigated roles of Smad2 in pancreas development and function.

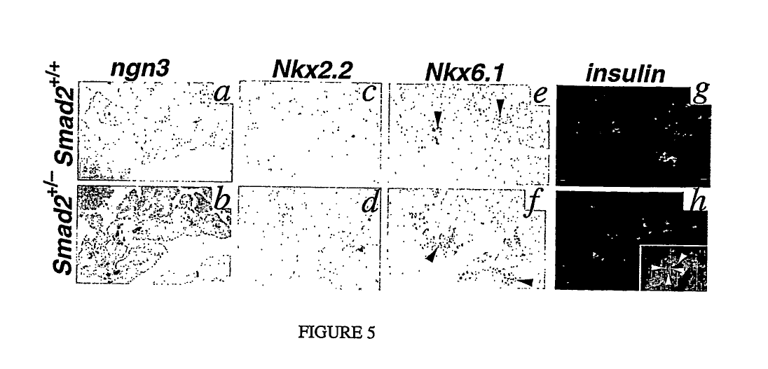

Mice heterozygous for Smad2 mutation are viable, and similar to ActRIIB−/− and ActRIIA+/−IIB+/− mutant mice, have (1) increased production of cells in the embryonic pancreas expressing neurogenin3, a marker of pancreatic islet precursors (2) increased numbers of immature pancreatic β-cells late in gestation (e.g., cells expressing the transcription factor Nkx6.1 but not insulin) (3) evidence of impaired islet maturation after birth, with islet hypoplasia and reduced β-cell mass, (4) normal islet architecture, without evidence of other organ malformations (5) impaired glucose tolerance, and (6) inadequate blood insulin levels. These results are encompassed in a manuscript in preparation (Harmon et al). Thus, our studies of pancreas development and glucose physiology in Smad2 mutant provide unexpected evidence for a molecular connection between TGF-β signaling and (I) regulation of islet precursor cell progenitor development and (II) β-cell maturation. These studies corroborate the studies of GDF-11 described below. Data are shown in FIG. 5.

### Example 3

### In Vitro Studies of Noggin, Chordin. GDF11 and GDF8 Activity on Mouse ES Cells During Formation of Insulin-producing Cell Clusters (IPCCs).

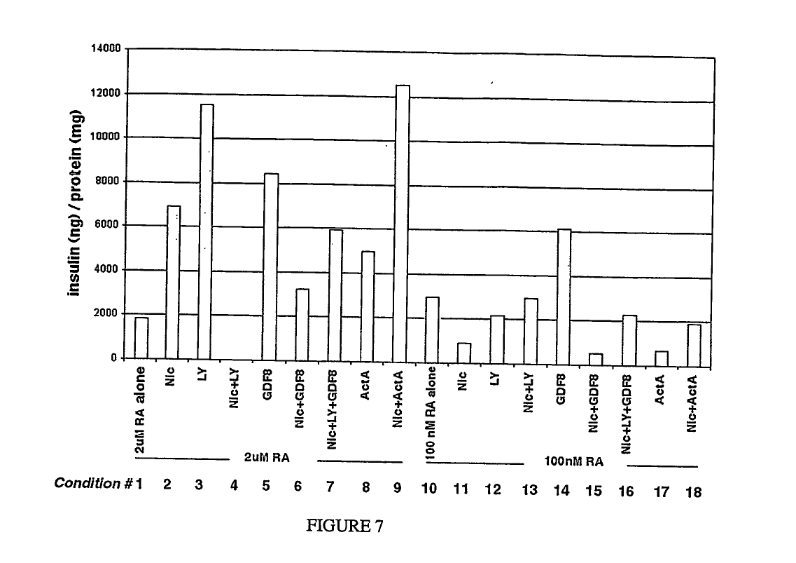

GDF8 (which is ˜80% identical to GDF11 in the mature region and has similar in vitro activities in a neural cell development assay as shown by published studies from the laboratory of Thomas Jessell, Columbia University) promotes insulin production in mouse ES cell-derived IPCCs. In duplicate experiments, we detect approximately 6000 ng insulin/mg protein produced by mouse embryoid bodies treated with a sequence of growth factors including GDF8. Data are shown in FIG. 7.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject inventions have been discussed, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method for increasing insulin production in a cell composition comprising stem cells, the method comprising contacting the cell composition with a BMP family member.

2. The method of claim 1, wherein the BMP family member is a member of a GDF8/GDF11 subfamily.

3. (canceled)

4. The method of claim 1, wherein the BMP family member binds to an ActRIIA and/or ActRIIB receptor.

5. The method of claim 4, wherein the BMP family member increases one or more of the following in a stem cell: (a) Smad2 phosphorylation (b) Smad3 phosphorylation and (c) expression of a gene that is positively regulated by Smad4.

6. (canceled)

7. The method of claim 1, wherein the cell composition comprises beta cell precursor cells.

8. A method for promoting the maturation of beta cell precursor cells, the method comprising contacting beta cell precursor cells with a BMP family member.

9. The method of claim 8, wherein the beta cell precursor cells express neuroD, nk×6.1 and/or nk33 2.2.

10. A method for increasing insulin production in a cell composition comprising stem cells, the method comprising contacting the cell composition with a substance that stimulates an ActRIIA and/or ActRIIB signaling pathway.

11. The method of claim 10, wherein the substance causes an increase in one or more of the following (a) Smad2 function (b) Smad3 function and (c) expression of a gene that is positively regulated by Smad4.

12. (canceled)

13. (canceled)

14. The method of claim 10, wherein the substance is a member of the GDF8/GDF11 subfamily.

15. The method of claim 10, wherein the substance is a nucleic acid comprising a nucleic acid sequence encoding a positive regulator of the ActRIIA and/or ActRIIB signaling pathway.

16. The method of claim 10, wherein the positive regulator is selected from the group consisting of: Smad2, Smad3, Smad4 and a GDF8/GDF11 subfamily member.

17. The method of claim 10, wherein the substance causes the inhibition of an inhibitor of the ActRIIA and/or ActRIIB signaling pathway.

18. (canceled)

19. A method for promoting the formation of beta cell precursor cells in a cell composition comprising stem cells, the method comprising contacting the cell composition with a substance that inhibits a BMP signaling pathway.

20. The method of claim 19, wherein the substance that inhibits a BMP signaling pathway is a secreted polypeptide that binds directly to a BMP family member.

21. The method of claim 19, wherein the substance that inhibits a BMP signaling pathway is selected from the group consisting of: a noggin, a chordin and a follistatin.

22. The method of claim 19, wherein the substance that antagonizes a BMP signaling pathway is a nucleic acid comprising a coding sequence for a polypeptide antagonist of a BMP signaling pathway.

23. A method for culturing a cell composition comprising stem cells to obtain an insulin producing cell composition, the method comprising:
a) culturing the cell composition in the presence of a substance that inhibits a BMP signaling pathway; and
b) culturing cells from (a) in the presence of a substance that stimulates a BMP signaling pathway.

24. The method of claim 23, wherein the substance that inhibits a BMP signaling pathway is a secreted polypeptide that binds directly to a BMP family member.

25. The method of claim 23, wherein the substance that inhibits BMP signaling is selected from the group consisting of: a noggin and a chordin.

26. The method of claim 23, wherein the substance that stimulates a BMP signaling pathway is a BMP family member.

27. The method of claim 26, wherein the BMP family member is a member of a GDF8/GDF11 subfamily.

28. (canceled)

29. The method of claim 23, wherein the stem cells are islet precursor cells.

30. (canceled)

31. A method for increasing insulin production in a subject, the method comprising administering to the subject a BMP family member in an amount sufficient to increase insulin production.

32. The method of claim 31, wherein the subject has been diagnosed with type I or type II diabetes.

33. The method of claim 31, wherein the BMP family member is a member of a GDF8/GDF11 subfamily.

34. A method for increasing the production of islet precursor cells in a subject, the method comprising administering to the subject a substance that inhibits a BMP signaling pathway.

35. The method of claim 34, wherein the substance that inhibits BMP signaling is selected from the group consisting of: a noggin, a chordin and a follistatin.

36. A method for increasing insulin production in a subject comprising administering to the subject a composition comprising insulin producing cells produced according to the method of any of claims 1, 8, 10 or 23.

37. A method for increasing the number of beta cell precursor cells in a subject comprising administering to the subject a composition comprising beta cell precursor cells produced according to the method of claim 19.

38.-46. (canceled)
